(19) United States
(12) Patent Application Publication
(10) Pub. No.: US 2023/0349003 A1
(43) Pub. Date: Nov. 2, 2023

(54) Methods for Salmonella Serovar Analysis and Differentiation

(71) Applicant: PathogenDx, Inc., Scottsdale, AZ (US)

(72) Inventors: Michael E. Hogan, Stony Brook, NY (US); Shaun Stice, Tucson, AZ (US); Melissa May, Tucson, AZ (US); Frederick Eggers, Sahuarita, AZ (US)

(73) Assignee: PathogenDx, Inc., Scottsdale, AZ (US)

(21) Appl. No.: 18/193,073

(22) Filed: Mar. 30, 2023

Related U.S. Application Data
(60) Provisional application No. 63/325,197, filed on Mar. 30, 2022.

(51) Int. Cl. C12Q 1/689; C12Q 1/6837

(52) U.S. Cl. CPC C12Q 1/689; C12Q 1/6837; C12R 2001/42

(57) ABSTRACT

Provided herein are methods for identifying and serotyping Salmonella spp. serovars. Primer pairs and nucleic acid probes complementary to signature determinants in specific serovars are utilized for PCR amplification and hybridization for differentiation among specific Salmonella spp. serovars in a single sample.

### CROSS-REFERENCE TO RELATED APPLICATIONS

This non-provisional application claims benefit of priority under 35 U.S.C. §119(e) of provisional application U.S. Serial No. 63/325,197, filed Mar. 30, 2022, the entirety of which is hereby incorporated by reference.

### BACKGROUND OF THE INVENTION

### Field of the Invention

The present invention relates to the field of pathogenic bacteria identification and serotyping. More particularly, the present invention relates to methods to identify and to differentiate among Salmonella serotypes using the presence or absence pattern of serovar-related gene markers via microarray analysis.

### Description of the Related Art

Salmonella species cause a wide variety of pathophysiological diseases in humans and farm animals which poses a threat to farmers and to food and agricultural industries. Salmonella infections are spread generally via ingestion of contaminated food and water, for example, meat products, poultry products, raw or undercooked eggs and dough, dairy products, fruits, leafy greens, raw sprouts, fresh vegetables, nut butters and spreads, pet foods and treats and by unhygenic handling of food and tools utilized to prepare the same.

The seriousness of the infection is dependent upon the serovar and the host. Thus, rapid screening of a sample to detect a Salmonella and to differentiate among Salmonella serovars would be beneficial to farmers and the food and agricultural industries as quality control and to identify the serovar in a subject exhibiting symptoms for diagnosis and treatment.

Thus, the prior art is deficient in means and methods of identifying and serotyping Salmonella species in a single sample. Specifically, the prior art is deficient in methods that enable differentiation among Salmonella serotypes quickly without crossover and with a high throughput via microarray analysis. Futhermore, the prior art is deficient in methods of detecting more than one serotype within a single sample, particularly a primary enrichment sample. The present invention fulfills this longstanding need and desire in the art.

### SUMMARY OF THE INVENTION

The present invention is directed to a method for detecting at least one Salmonella sp. serovar in a sample. In this method, a sample is obtained and DNA is extracted therefrom. An amplification reaction is performed on the at least one DNA using at least one fluorescently-labeled primer pair selective for the at least one Salmonella sp. serovar to generate fluorescently-labeled serovar DNA amplicons. The fluorescently-labeled serovar DNA amplicons are hybridized to a plurality of nucleic acid probes each having a sequence corresponding to a sequence determinant in the Salmonella sp. serovar DNA and each attached to a microarray. The microarray is washed at least once and the microarray is imaged to detect at least one fluorescent signal from the fluorescently-labeled serovar DNA amplicons, thereby detecting the Salmonella sp. serovar in the sample.

The present invention is further directed to a method for serotyping Salmonella in a sample matrix. In this method, a sample is obtained from the sample matrix and total DNA is isolated therefrom. An amplification reaction is performed on the total DNA using a plurality of fluorescently-labeled primer pairs selective for all Salmonella serovars to generate fluorescently-labeled serovar DNA amplicons. The fluorescently-labeled serovar DNA amplicons are hybridized to a plurality of nucleic acid probes each having a sequence complementary to a sequence determinant in the Salmonella DNA that discriminates among the Salmonella serovars, where each of the nucleic acid probes is attached at a specific position on a microarray support. The microarray is washed at least once. The microarray support is imaged to detect at least one fluorescent signal from the hybridized fluorescently-labeled serovar DNA amplicons and the specific position of the fluorescent signal on the microarray support is correlated to a specific Salmonella serotype.

The present invention is directed further to a method for testing a food product for the presence of Salmonella. In this method, a selective media enrichment of a food matrix associated with the food product. A bacterial pool is extracted therefrom and total DNA is isolated from the bacterial pool. At least one amplification reaction is performed on the total DNA using at least one fluorescently-labeled primer pair selective for at least one Salmonella sp. serovar gene target and a generic Salmonella sp. marker to generate fluorescently-labeled serovar DNA amplicons. The fluorescently-labeled serovar DNA amplicons are hybridized to nucleic acid probes each having a sequence complementary to a gene sequence determinant in at least one Salmonella sp. DNA that discriminates among the Salmonella sp. serovars, where each of said nucleic acid probes attached at a specific position on a microarray support. The microarray is washed at least once and the microarray support is imaged to detect at least one fluorescent signal from the hybridized fluorescently-labeled serovar DNA amplicons, where the specific position of the fluorescent signal and a target gene profile on the microarray support identifies a specific Salmonella sp. serotype in the food product.

These and other features, aspects, and advantages of the embodiments of the present disclosure will become better understood when the following detailed description is read with reference to the accompanying drawings.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1F are bar graphs illustrating the detection of Salmonella enterica serovars Enteritidis (FIG. 1A), Heidelberg (FIG. 1B), Infantis (FIG. 1C), Newport (FIG. 1D), Typhimurium (FIG. 1E), and Javiana (FIG. 1F).

FIGS. 2A-2C are bar graphs illustrating the detection of extracted gDNA from raw poultry tender nBPW enrichment (for 20 hrs) naturally contaminated with strains of Salmonella enterica subsp. Enterica serovar Saintpaul and Montevideo (FIG. 2A) and artificially inoculated with Enteritidis and Typhimurium (FIG. 2B) and artificially inoculated with Typhimurium (FIG. 2C).

### DETAILED DESCRIPTION OF THE INVENTION

The articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

The term “or” in the claims refers to “and/or” unless explicitly indicated to refer to alternatives only or the alternatives are mutually exclusive, although the disclosure supports a definition that refers to only alternatives and “and/or”.

The terms “comprise” and “comprising” are used in the inclusive, open sense, meaning that additional elements may be included.

The term “including” is used herein to mean “including, but not limited to”. “Including” and “including but not limited to” are used interchangeably.

As used herein, the term “about” refers to a numeric value, including, for example, whole numbers, fractions, and percentages, whether or not explicitly indicated. The term “about” generally refers to a range of numerical values (e.g., +/- 5-10% of the recited value) that one of ordinary skill in the art would consider equivalent to the recited value (e.g., having the same function or result). In some instances, the term “about” may include numerical values that are rounded to the nearest significant figure.

As used herein, the terms “microarray” and “microarray support” are interchangeable.

As used herein, the term “subject” refers to a human or other mammal, for example, but not limited to, a farm animal.

In one embodiment of this invention, there is provided a method for identifying at least one Salmonella sp. serovar in a sample, comprising obtaining the sample, extracting DNA therefrom; performing an amplification reaction on the DNA using at least one fluorescently-labeled primer pair selective for the at least one Salmonella sp. serovar to generate fluorescently-labeled serovar DNA amplicons; hybridizing the fluorescently-labeled serovar DNA amplicons to a plurality of nucleic acid probes each having a sequence corresponding to a sequence determinant in the Salmonella sp. serovar DNA and each attached to a microarray; washing the microarray at least once; and imaging the microarray to detect at least one fluorescent signal from the fluorescently-labeled serovar DNA amplicons, thereby detecting the at least one Salmonella sp. serovar in the sample.

In this embodiment, the sample may be a primary enrichment of a sample matrix, a rinsate of the sample matrix or a swab of the sample matrix. In this embodiment, a representative Salmonella sp. may be Salmonella enterica. In an aspect of this embodiment, a representative Salmonella enterica serovar may be selected from the group including but not limited to Enteritidis, Heidelberg, Infantis, Newport, Typhimurium, Javiana, I 4,[5],12:i:-, Muenchen, Saintpaul, Montevideo, Braenderup, Oranienburg, and Thompson.

In this embodiment and aspect thereof, the primer pair may comprise nucleotide sequences selected from the group consisting of SEQ ID NOS: 1 and 2, SEQ ID NOS: 3 and 4, SEQ ID NOS: 5 and 6, SEQ ID NOS: 7 and 8, SEQ ID NOS: 9 and 10, SEQ ID NOS: 11 and 12, SEQ ID NOS: 13 and 14, SEQ ID NOS: 15 and 16, SEQ ID NOS: 17 and 18, SEQ ID NOS: 19 and 20, SEQ ID NOS: 21 and 22, SEQ ID NOS: 23 and 24, SEQ ID NOS: 25 and 26, and SEQ ID NOS: 27 and 28. Also in this embodiment and aspect thereof, the nucleic acid probes may comprise nucleotide sequences selected from the group consisting of SEQ ID NOS: 31-60. In addition, the sample may be obtained from a subject, a farm animal, a plant, a food product, a processing surface, or water or a swab thereof.

In another embodiment of this invention, there is provided a method for serotyping a Salmonella in a sample matrix, comprising obtaining a sample from the sample matrix; isolating total DNA therefrom; performing an amplification reaction on the total DNA using a plurality of fluorescently-labeled primer pairs selective for all Salmonella serovars to generate fluorescently-labeled serovar DNA amplicons; hybridizing the fluorescently-labeled serovar DNA amplicons to a plurality of nucleic acid probes each having a sequence complementary to a sequence determinant in the Salmonella DNA that discriminates among the Salmonella serovars, each of said nucleic acid probes attached at a specific position on a microarray support; washing the microarray support at least once; imaging the microarray support to detect at least one fluorescent signal from the hybridized fluorescently-labeled serovar DNA amplicons; and correlating the specific position of the fluorescent signal on the microarray support to a specific Salmonella serotype.

In this embodiment the sample matrix is processed with an enrichment culture or without an enrichment culture. In an aspect of this embodiment the sample matrix is processed without the enrichment culture, where the sample comprises a rinsate of the sample matrix or a swab of the sample matrix. In this embodiment and aspect thereof the sample may be obtained as described supra.

In this embodiment, representative Salmonella serotypes include but are not limited to Salmonella enterica Enteritidis, Salmonella enterica Heidelberg, Salmonella enterica Infantis, Salmonella enterica Newport, Salmonella enterica Typhimurium, Salmonella enterica Javiana, Salmonella enterica I 4,[5],12:i:-, Salmonella enterica Muenchen, Salmonella enterica Saintpaul, Salmonella enterica Montevideo, Salmonella enterica Braenderup, Salmonella enterica Oranienburg, or Salmonella enterica Thompson. Also, the plurality of primer pairs and the plurality of nucleic acid probes comprise nucleotide sequences as described supra.

In yet another embodiment of this invention, there is provided a method for detecting the presence of Salmonella, comprising obtaining a selective media enrichment of a food matrix associated with the food product; extracting a bacterial pool therefrom; isolating total DNA from the bacterial pool; performing at least one amplification reaction on the total DNA using at least one fluorescently-labeled primer pair selective for at least one Salmonella sp. serovar gene target and a generic Salmonella sp. marker to generate fluorescently-labeled serovar DNA amplicons; hybridizing the fluorescently-labeled serovar DNA amplicons to nucleic acid probes each having a sequence complementary to a gene sequence determinant in at least one Salmonella sp. DNA that discriminates among the Salmonella sp. serovars, each of said nucleic acid probes attached at a specific position on a microarray support; washing the microarray at least once; imaging the microarray support to detect at least one fluorescent signal from the hybridized fluorescently-labeled serovar DNA amplicons, wherein the specific position of the fluorescent signal and a target gene profile on the microarray support identifies a specific Salmonella sp. serotype in the food product.

In this embodiment, the Salmonella sp., the Salmonella sp. serovar, the plurality of primer pairs, and the plurality of nucleic acid probes are as described supra. Further in this embodiment, the generic Salmonella sp. marker may be invA. In addition, the food product may be a product from a farm animal, a cultivated plant or water used in the raising or cultivation thereof or from a processing surface for the food product. Alternatively, the food product may be a processed food product.

Provided herein are methods for identifying and serotyping Salmonella species, including, but not limited to, Salmonella enterica and associated serovars or serotypes. The present invention is differentiated from other methods for serotyping Salmonella by its ability to include multiple gene targets that are highly correlated with specific Salmonella serotypes. The method enables simultaneous detection of both the presence and the absence of a gene marker and thus the Salmonella species in a raw, mixed sample based on the fluorescence or lack thereof emitted after hybridization of the serovar amplicons generated via amplification of the DNA in a sample. Some serovar strains have more than one gene marker present in their genome. In this situation, the presence/absence profile of the specific gene markers is determined by experimentation. This presence/absence profile is translated by the associated Augary software to successfully identify the serovar in question. Table 1 lists Salmonella spp. serovars, the gene targets and their associated publications.

TABLE 1

Microarray-based serotyping assay gene markers

| | | |
|---|---|---|
| Species / Serovar | Gene Marker | Reference |
| Salmonella spp. | invA | Rahn et al. 1992 |
| Enteritidis | safA | Maurischat et al. 2015 |
| Typhimurium | STM4200 | Heymans et al. 2018 |
| Newport | hypothetical protein | Bugarel et al. 2017 |
| Javiana | hypothetical protein | Zhang et al. 2019 |
| I 4,[5],12:i:- | fljB | Maurischat et al. 2015 |
| Heidelberg | type II restriction enzyme | Afroj et al. 2017 |
| Muenchen | nucleotide-binding protein | Zhang et al. 2019 |
| Saintpaul | dndE | Zhang et al. 2019 |
| Montevideo | hypothetical protein | Zhang et al. 2019 |
| Infantis | SIN_02055 | Yang et al. 2021 |
| Braenderup | hypothetical protein | Zhang et al. 2019 |
| Oranienburg | hypothetical protein | Zhang et al. 2019 |
| Thompson | hypothetical protein | Zhang et al. 2019 |

Samples may be obtained from a media enrichment of a sample matrix obtained from, but not limited to, a human subject, a farm animal, a plant, a food product or food stuff, water. The media enrichment may be a selective enrichment. Alternatively, the sample may be obtained without enrichment, for example, but not limited to, a rinsate, for example, a poultry rinse, or from a swab. In the methods provided herein, a bacterial pool may be isolated from the enriched sample matrix and total DNA extracted or isolated therefrom without first isolating single colonies. In certain cases, where the level of Salmonella contamination is high, the duration of enrichment culture may be reduced or eliminated entirely, such that DNA may be extracted directly from a rinsate or a swab with limited or no prior culture.

In a non-limiting example, the food product or food stuff may be a product from a farm animal, a cultivated plant or water used to raise the farm and/or cultivate the plant. Another non limiting example is a swab obtained from a human subject, a farm animal, a plant, a food product or food stuff or a processing surface, such as used in the processing and production of the food.product or plant.

The following examples are given for the purpose of illustrating various embodiments of the invention and are not meant to limit the present invention in any fashion.

### Example 1

### PCR for Amplifying Salmonella Enterica DNA

PCR is performed on purified Salmonella enterica at the cycling conditions shown in Table 2.

TABLE 2

PCR Cycling Conditions
PCR

| Steps | Temp. | Time | Cycles |
|---|---|---|---|
| 1 | 95° C. | 4 minutes | 1 |
| 2 | 95° C. | 30 seconds | 40 |
| 3 | 55° C. | 30 seconds | |
| 4 | 72° C. | 1 minute | |
| 5 | 72° C. | 7 minutes | 1 |
| 6 | 15° C. | ∞ | 1 |

Singleplex or multiplex PCR is performed using at least one fluorescently labeled primer pair for each serovar DNA. Table 3 lists non-limiting examples of primer pairs. The primers each may have a 5′-terminal fluorescent label, for example, but not limited to, the cyanine fluorophores CY3 or CY5.

TABLE 3

S. enterica serovar primers

| | | |
|---|---|---|
| S. enterica Serovar | SEQ ID NO | Primer Sequence |
| Enteritidis | SEQ ID NO: 1 Forward primer | TTTTTTGGGGCATTGGTATCAAAG |
| | SEQ ID NO: 2 Reverse primer | /5Cy3/TTTTTGGTTGCTAACACGACACTG |
| Typhimurium | SEQ ID NO: 3 Forward primer | TTTTTCACCTGATATAGAGTCCAA |
| | SEQ ID NO: 4 Reverse primer | /5Cy3/TTTTTTTATAGATGTTGTCGCCAA |
| Newport | SEQ ID NO: 5 Forward primer | TTTTAATGGCTGGTAGCCTGTTCG |
| | SEQ ID NO: 6 Reverse primer | /5Cy3/TTTAGGGAAAGCAAGGAACAGTAG |
| Javiana | SEQ ID NO: 7 Forward primer | TTTTAAAACGCCATGAGCTTTCTC |
| | SEQ ID NO: 8 Reverse primer | /5Cy3/TTTTGTGCGTTGATAAGTTGTGCT |
| Monophasic Typhimurium | SEQ ID NO: 9 Forward primer | TTTTTTGGTGCTGTTAGCAGAC |
| | SEQ ID NO: 10 Reverse primer | /5Cy3/TTTTCAACACTAACAGTCTGTCG |
| Heidelberg | SEQ ID NO: 11 Forward primer | TTTTGCAGTTCATTCGCTTTGTCG |
| | SEQ ID NO: 12 Reverse primer | /5Cy3/TTTCGGAAAATACGTCTCATGTCC |
| Saintpaul | SEQ ID NO: 13 Forward primer | TTTGAATGGTACTTAGCCGTCAGA |
| | SEQ ID NO: 14 Reverse primer | /5Cy3/TTTCTTCTTACTATCCCGCTCAGG |
| Montevideo | SEQ ID NO: 15 Forward primer | TTTATGAATGTCGCCTATCCTGAC |
| | SEQ ID NO: 16 Reverse primer | /5Cy3/TTTTCTTCTGACGGATAATGTGCA |
| Infantis | SEQ ID NO: 17 Forward primer | TTTTGGTCGAGATGGGTATGTAGC |
| | SEQ ID NO: 18 Reverse primer | /5Cy3/TTTTCAGGAGTTCCTGCGCAACCA |
| Barenderup | SEQ ID NO: 19 Forward primer | TTTGCTAATGACTTCGGAGCAAAG |
| | SEQ ID NO: 20 Reverse primer | /5Cy3/TTTTTCACTTGGGTTAAAGCGTG |
| Oranienberg | SEQ ID NO: 21 Forward primer | TTTTGCTGAGATTGTGATTCCACC |
| | SEQ ID NO: 22 Reverse primer | /5Cy3/TTTTCGCTGTTCTAACCTTGAGGA |
| Thompson | SEQ ID NO: 23 Forward primer | TTTATTCGGCGAGCCAATATTTTC |
| | SEQ ID NO: 24 Reverse primer | /5Cy3/TTTATCATTTGTACCCTGATGCCA |
| Salmonella spp. (invA) | SEQ ID NO: 25 Forward primer | TTTATCGTTATTACCAAAGGTTCAG |
| | SEQ ID NO: 26 Reverse primer | /5Cy3/TTCCTTTCCAGTACGCTTCGCCGTTCG |
| Muenchen | SEQ ID NO: 27 Forward primer | TTTCGTATGCAGATCGAAGATCCT |
| | SEQ ID NO: 28 Reverse primer | /5Cy3/TTTATAACTGTGTTAGCCGTTCCA |
| Positive Control | SEQ ID NO: 29 Forward primer | TTTACCTGATGGCCCTCATTAGTCCTTG |
| | SEQ ID NO: 30 Reverse primer | /5Cy3/TTTGACGGCTGTCAGCGCCTGTGCTTC |

### Example 2

### Hybridization of S. Enterica Serovar Amplicons

The S. enterica serovar amplicons are hybridized to a microarray or a microarray support, such as, but is not limited to, a microarray with a functionalized solid surface, to which a plurality of S. enterica nucleic acid probes are directly or indirectly covalently attached. The attachment site correlates to a specific serovar nucleic acid sequence. The nucleic acid probes may be indirectly covalently attached via linker, for example, a bifunctional oligonucleotide linker, such as, but not limited to, the oligothymidine linker OLIGO-T, which is covalently attached at one terminal nucleotide to the functionalized and covalently crosslinked to at least one nucleic acid probe at the other terminus in a 3-dimensional lattice formation.

Table 4 lists non-limiting examples of nucleic acid probes selective for sequence determinants complementary to specific S. enterica serovar DNA.

TABLE 4

S. enterica serovar probes

| | | |
|---|---|---|
| S. enterica Serovar | SEQ ID NO | Probe Sequence |
| Enteritidis | SEQ ID NO: 31 | TTTTTCTCCTCCCATTCCACATTTGCGTTTTT |
| | SEQ ID NO: 32 | TTTTTGCTCCTCCCATTCCACATTTGCTTTTT |
| Typhimurium | SEQ ID NO: 33 | TTTTGAACAATGCCTCCCGCTCCTCCTGCCTT |
| | SEQ ID NO: 34 | TTTTATTCTTGACTGAACAATGCCTCCTTT |
| Javiana | SEQ ID NO: 35 | TTTTCTCCTGTGATAAAAGTTGTCTTGCTCTTT TT |
| | SEQ ID NO: 36 | TTTTGGGGTAAAAACAAGAAAAATCTCCCTTTT |
| Monophasic Typhimurium | SEQ ID NO: 37 | TTTTTCGGACTGGGATTTGTTCAGGTTATTTTT |
| | SEQ ID NO: 38 | TTTTCTTGATACGCAGACCAGAAGACAGTTTTT |
| Heidelberg | SEQ ID NO: 39 | TTCCCAGTAGTCCATCACCCAGCGCAGTCTTT |
| | SEQ ID NO: 40 | TTTTTTAGGTACTGTTATCTTCGAGGCGTTTTT |
| Muenchen | SEQ ID NO: 41 | TTTTACACCTCTTTTAGATTACCTAGATTTT |
| | SEQ ID NO: 42 | TTTTTCGTATGCAGATCGAAGATCCTCTTTT |
| Saintpaul | SEQ ID NO: 43 | TTTTCTAGTGGAGAGTGAGTTTCGCTATTCTTT |
| | SEQ ID NO: 44 | TTTCTCAAAGGATATACGGGGATTACACCTTTT |
| Montevideo | SEQ ID NO: 45 | TTTTAACCTAAACAGAATAACAAAACATTTTT |
| | SEQ ID NO: 46 | TTTTCAAAACCACCTTTAGTACATCTCCATTTT |
| Infantis | SEQ ID NO: 47 | CTCGTTCACCTAAGAGAATTATTGTAAAAGTCT |
| | SEQ ID NO: 48 | TTCCCACCTAAGAGAATTATTGTAAAAGTCTT |
| Braenderup | SEQ ID NO: 49 | TTTTGATTGCAGGAGAATTGCGTATGGTTTT |
| | SEQ ID NO: 50 | TTTTAGAGAGTGCGGACATTTATAGCTCTCTTT |
| Oranienberg | SEQ ID NO: 51 | TTTTTGTGATTCCACCAGAAGAGTTTGTTTT |
| | SEQ ID NO: 52 | TTTTTGGCGTAGTATTAAAAACCCCTTTTT |
| Thompson | SEQ ID NO: 53 | TTTCTTGGTGCGAGAGGATTAAAAACACTTTTT |
| | SEQ ID NO: 54 | TTTTTAAGTTACTTCGTAATTCCACTCTTTT |
| Newport | SEQ ID NO: 55 | TTCTTGCACTGGGAACAATTTCTGGCTATTTT |
| | SEQ ID NO: 56 | TTCTCACTGGGAACAATTTCTGGCTACATTTT |
| Negative | SEQ ID NO: 57 | TTTTTTCTACTACCTATGCTGATTCACTCTTTTT |
| Positive Control | SEQ ID NO: 58 | TTTATTCTGCTCTTATCTTGGATTTTATTT |
| invA | SEQ ID NO: 59 | TTTTTTTATTGATGCCGATTTGAAGGCCTTTTT T |
| | SEQ ID NO: 60 | TTTTTTTCTGATGCCGATTTGAATTTTTTT |

### Example 3

### Microarray Discrimination Among Salmonella Enterica Serovars

Raw chicken tenders were purchased from the local grocery store and were rinsed with Buffered Peptone Water (BPW). 30 mL of the BPW rinse was added to 30 mL of sterile BPW in a sample container, and vortexed. Each Salmonella strain in question was spiked into the sample enrichment at a concentration of 5 CFU/mL. The sample was incubated at 35° C. for 20 hours. The DNA was extracted from the primary enrichment using a commercial magbead extraction kit (Zymo Quick DNA/RNA Viral Magbead kit), briefly 200 µL of the overnight enrichment was added to 20 µL of DNA/RNA shield and mixed well. 400 µL of Viral DNA/RNA buffer was added to the sample and mixed. 10 µL of MagBinding Beads were added to the sample and mixed at 800 RPM for 10 minutes. The beads were pulled down with a magnetic stand and subsequent washes and elution was carried out. The sample was eluted in 30 µL. 5 µL of the extraction was used in the subsequent PCR reaction. The PCR reaction was set up by adding 5 µL of template, 25 µL of master mix, 0.4 µL of positive control, and 17.6 µL of molecular grade water, and 2 µL of the Cy3 labeled primer mix for a total volume of 50 µL. The primer sequences Table 3 (SEQ ID NOS: 1-30). The PCR reaction was placed in a thermocycler and run with the following conditions: 95C 4 min; 95° C. 30 sec, 55C 30 sec, 72° C. 1 min 45 cycles; 72° C. 7 min, 15° C. hold (Table 2).

Following PCR the microarray plat with the probes (Table 4, SEQ ID NOS: 31-60) was prepared for hybridization. 200 µL of water was added to the well and aspirated, an additional 200 µL of water was added to the well and incubated at room temperature for 5 min. The water was aspirated and 200 µL of the prehybridization buffer was added and incubated for 5 min and aspirated. The hybridization solution was prepared and 18 µL was added to the 50 µL PCR reaction. The 68 µL of PCR/hybridization solution was added to the well and incubated at room temperature for 30 min and aspirated. 200 µl of was solution was added and aspirated. 200 µL of wash solution was added again an incubated for 10 min at room temperature. One final wash was conducted, and the plate was dried for 5 min in a plate spinner. The plate was imaged using the default imaging conditions for PathogenDx assays using the plate scanner.

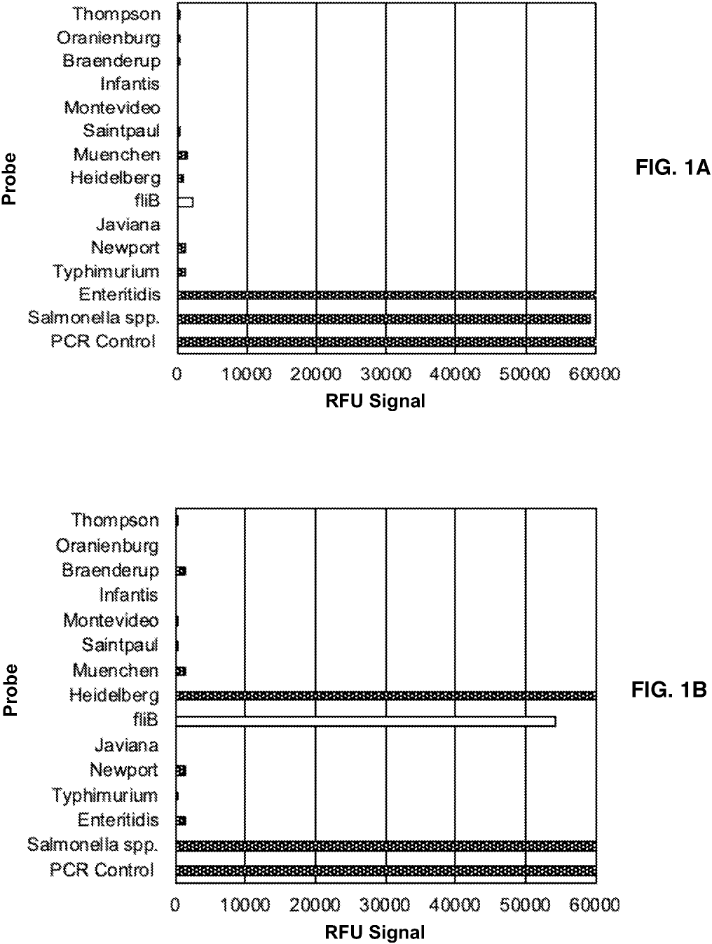

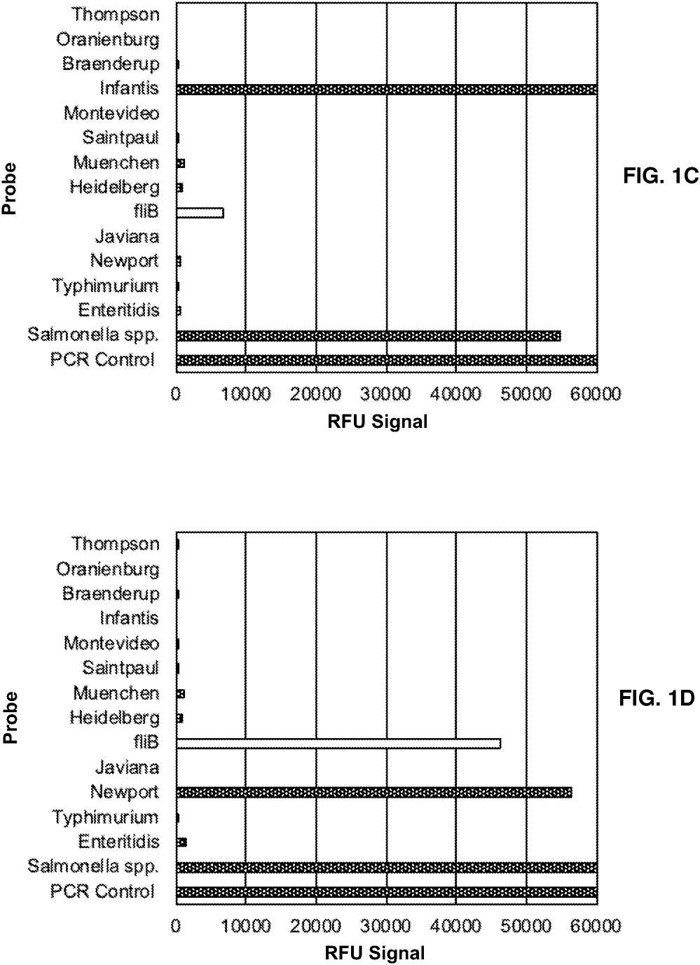

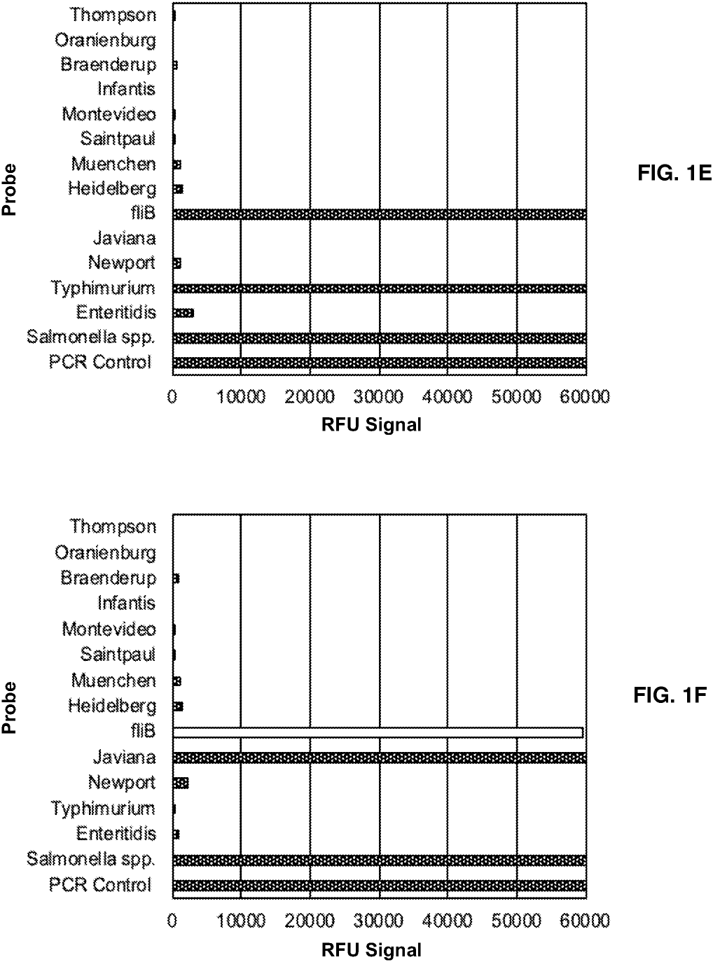

FIGS. 1A-1F demonstrate that a specific S. enterica serovar may be detected from among a plurality of serovars via microarray assay. Table 4 lists the RFU probe values for each of six serovars for various strains of S. enterica.

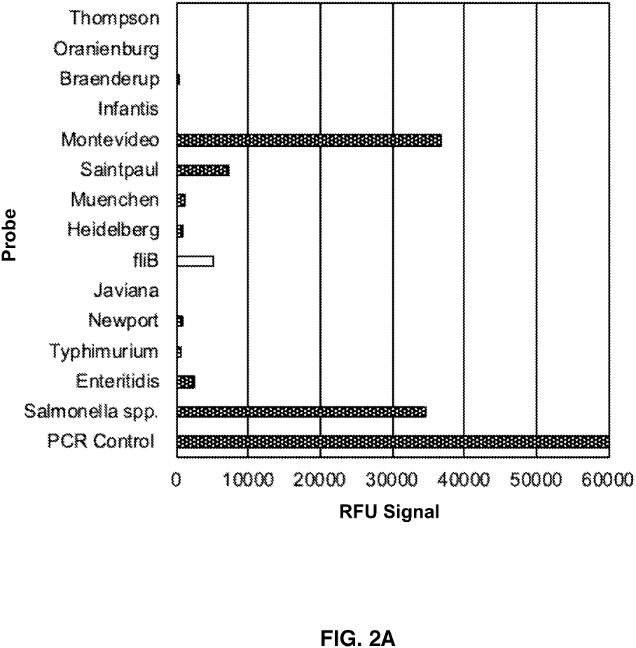

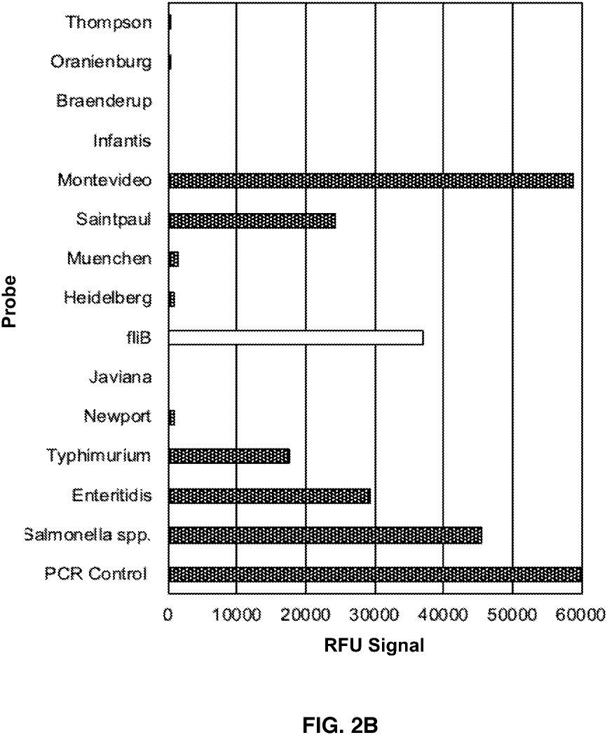

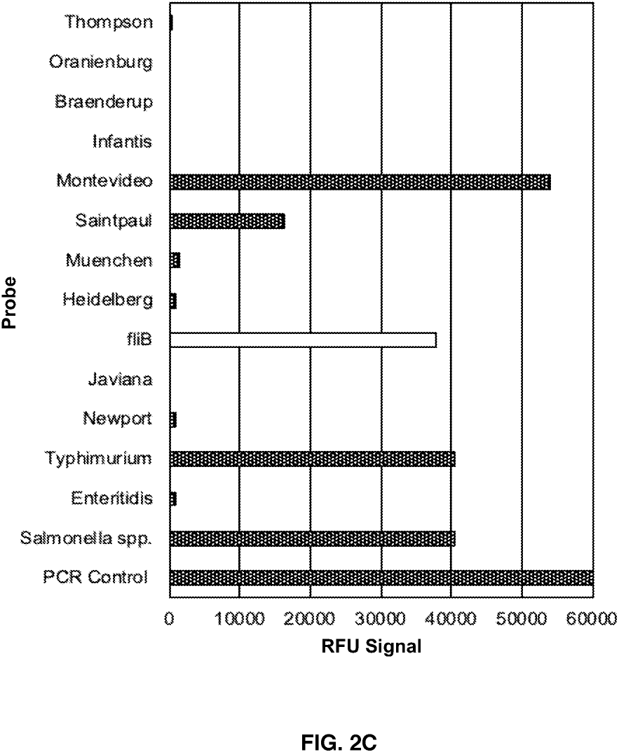

FIGS. 2A-2C demonstrate that multiple Salmonella serotypes can be detected from a single primary poultry enrichment contaminated with multiple strains of various serotypes.

Table 5 lists the gene marker look-up table for the serovars reported in this assay. Tables 6A-6C list the RFU probe values for each of thirteen serovars for various strains of S. enterica.

TABLE 5

Lookup Table for Serovar

| | | Probe output | | | | | | | | | | | | |
|---|---|---|---|---|---|---|---|---|---|---|---|---|---|---|
| | | Pr. Enteritidis | Pr.Typhimurium | Pr. Javiana | Pr.fliB | Pr. Heidelburg | Pr.Muenchen | Pr.Saintpaul | Pr.Montevideo | Pr.Infantis | Pr. Braenderup | Pr. Oranienberg | Pr.Thompson | Pr. Newport |
| Serotype reported by software | Enteritidis | + | - | - | +/- | - | - | - | - | - | - | - | - | - |
| | Typhimurium* | - | + | - | + | - | - | - | - | - | - | - | - | - |
| | Javiana | - | - | + | +/- | - | - | - | - | - | - | - | - | |
| | I 4,[5],12:i:- * | - | + | - | - | - | - | - | - | - | - | - | - | - |
| | Heidelberg | - | - | - | +/- | + | - | - | - | - | - | - | - | - |
| | Muenchen* | - | - | - | +/- | - | + | - | - | - | - | - | - | +/- |
| | Saintpaul | - | - | - | +/- | - | - | + | - | - | - | - | - | - |
| | Montevideo | - | - | - | +/- | - | - | - | + | - | - | - | - | - |
| | Infantis | - | - | - | +/- | - | - | - | - | + | - | - | - | - |
| | Braenderup | - | - | - | +/- | - | - | - | - | - | + | - | - | - |
| | Oranienberg* | - | - | - | +/- | - | - | - | +/- | - | | + | - | - |
| | Thompson | - | - | - | +/- | - | - | - | - | - | - | - | + | - |
| | Newport | - | - | - | +/- | - | - | - | - | - | - | - | - | + |

TABLE 6A

Detection of single serovar

| Organism and Strain | RFU Probe Value | | | | |
|---|---|---|---|---|---|
| | Probe Enteridis | Probe Heidelberg | Probe Infantis | Probe Newport | Probe Typhimurium |
| Muenchen MZ1478 | 977 | 959 | -844 | 48838 | 422 |
| Infantis DUP-103 | 717 | 785 | 61339 | 678 | 98 |
| I 4,[5],12:i:-USDA1 | 869 | 833 | -919 | 938 | 62548 |
| Montevideo G4639 | 5876 | 3032 | -985 | 1399 | 175 |
| Enteritidis NCTC 4444 | 63280 | 1026 | -961 | 1261 | 1209 |
| Heidelberg [16] | 1414 | 63894 | -951 | 1375 | 166 |
| Javiana ETS 146 | 891 | 1558 | -854 | 2163 | 331 |
| Newport NCTC 129 | 1399 | 898 | -980 | 56407 | 236 |
| Braenderup NCTC 5750 | 834 | 1570 | -794 | 1773 | 458 |
| Typhimurium CDC 6516-60 | 2956 | 1411 | -833 | 1064 | 63735 |
| Oranienberg E1093 | 5336 | 773 | -1005 | 1542 | 264 |
| Saintpaul 127 | 977 | 6633 | -867 | 1107 | 305 |
| Thompson BAA-3141 | 1178 | 895 | -816 | 1101 | 167 |
| Neg Control | 1083 | 1004 | -887 | 800 | 253 |

TABLE 6B

Detection of single serovar

| Organism and Strain | RFU Probe Value | | | |
|---|---|---|---|---|
| | Probe Oranienberg | Probe Thompson | Probe Braenderup | Probe Muenchen |
| Muenchen | 0 | -173 | 21 | 653 |
| MZ1478 | | | | |
| Infantis DUP-103 | -315 | -10 | -80 | 418 |
| I 4,[5],12:i:-USDA1 | -105 | 81 | -138 | 213 |
| Montevideo G4639 | 11 | 398 | 58298 | 366 |
| Enteritidis NCTC 4444 | 267 | 231 | -196 | 404 |
| Heidelberg [16] | -114 | 293 | 113 | 1314 |
| Javiana ETS 146 | -181 | -264 | 349 | 977 |
| Newport NCTC 129 | -396 | 205 | 164 | 289 |
| Braenderup NCTC 5750 | -97 | -89 | -126 | 45498 |
| Typhimurium CDC 6516-60 | -98 | 411 | 209 | 688 |
| Oranienberg E1093 | 63723 | 777 | 55914 | 354 |
| Saintpaul 127 | -258 | 521 | 61 | 552 |
| Thompson BAA-3141 | -134 | 48489 | 0 | 363 |
| Neg Control | -340 | 350 | -112 | 265 |

TABLE 6C

Detection of single serovar

| Organism and Strain | RFU Probe Value | | | |
|---|---|---|---|---|
| | Probe Javiana | Probe Saintpaul | Probe Infantis | Probe fliB |
| Muenchen MZ1478 | 39218 | -498 | 356 | 46329 |
| Infantis DUP-103 | 1299 | -696 | 85 | 6841 |
| I 4,[5],12:i:-USDA1 | 1278 | -345 | 297 | 736 |
| Montevideo G4639 | 1330 | -524 | 34 | 31196 |
| Enteritidis NCTC 4444 | 1420 | -449 | 110 | 2151 |
| Heidelberg [16] | 1416 | -519 | 105 | 54332 |
| Javiana ETS 146 | 1203 | 61894 | 185 | 59663 |
| Newport NCTC 129 | 1300 | -697 | 64 | 46328 |
| Braenderup NCTC 5750 | 1480 | -381 | 218 | 63762 |
| Typhimurium CDC 6516-60 | 1253 | -356 | 380 | 60642 |
| Oranienberg E1093 | 1040 | -548 | 9 | 13456 |
| Saintpaul 127 | 1447 | -464 | 61621 | 53979 |
| Thompson BAA-3141 | 1471 | -454 | 197 | 53523 |
| Neg Control | 1513 | -512 | 372 | 1075 |

### Example 4

### Detection of Salmonella Serovars on Surfaces Without Enrichment Culture

The present invention is used to detect Salmonella and its serovar subtypes in environmental samples obtained as a surface swab via the method of Katchman et al. (J AOAC Int. 105(5):1390-1407, Sept. 6, 2022). The method samples surfaces with a swab to collect bacteria, including salmonella, then prepare bacterial DNA from the swab for microarray analysis comprising: centrifugation of a swab eluate to harvest the cells, then an enzyme treatment to remove extra-cellular DNA, followed by cell lysis, 2-step tandem PCR of the lysate, followed by microarray hybridization and washing. The modifications made to the above published method, as used in this Example lie in the serover specific PCR primers used in the assay (Table 3) and the serovar specific microarray probes used in the microarray assay (Table 4).

Briefly, a surface sample is collected with an environmental swab such as WorldBio PUR-Blue™ Swabs in a 5 mL tube of Hi-Cap broth (BLU-HC-P). Upon swabbing of the surface, the swab is placed back in the tube for shipping and transport. On return to a lab, the swab is vortexed in the transport medium. 1 ml of the transport medium is then centrifuged to pellet bacterial cells and cellular debris. The pellet is then treated with an enzyme kit which degrades the cell free bacterial DNA and retains cellular DNA for analysis. The resulting cellular pellet is then lysed with heat treatment. 2 µL of the lysate is then used directly for PCR amplification.

DNA in the lysate is amplified via a 2-step tandem Polymerase Chain Reaction (PCR) which allows bacteria in the sample to be analyzed without prior enrichment culture. The enhanced sensitivity of the 2-step PCR reaction obviates the need for culture based amplification based on cell growth. The final CY3 labeled PCR product is used without amplicon clean-up, quantitation, or normalization prior to hybridization on the microarray containing the serovar specific probes of Table 4). The hybridized and washed microarray is then imaged to yield a CY3 hybridization pattern distributed among the probe spots. The PathogenDx software analysis tool, Augury©, automatically finds the hybridized spots in the image and then calculates the median CY3 intensity of each hybridized spot. The resulting hybridization pattern is thus used to define which salmonella serovars are present in the surface derived sample, exactly as was shown in Example 3, for the corresponding products of culture based enrichment.

What is claimed is:

1. A method for identifying at least one Salmonella sp. serovar in a sample, comprising:
obtaining the sample,
extracting DNA therefrom;
performing an amplification reaction on the DNA using at least one fluorescently-labeled primer pair selective for the at least one Salmonella sp. serovar to generate fluorescently-labeled serovar DNA amplicons;
hybridizing the fluorescently-labeled serovar DNA amplicons to a plurality of nucleic acid probes each having a sequence corresponding to a sequence determinant in the Salmonella sp. serovar DNA and each attached to a microarray;
washing the microarray at least once; and
imaging the microarray to detect at least one fluorescent signal from the fluorescently-labeled serovar DNA amplicons, thereby detecting the at least one Salmonella sp. serovar in the sample.

2. The method of claim 1, wherein the sample is a primary enrichment of a sample matrix, a rinsate of the sample matrix or a swab of the sample matrix.

3. The method of claim 1, wherein the Salmonella sp. is Salmonella enterica.

4. The method of claim 3, wherein the Salmonella enterica serovar is selected from the group consisting of Enteritidis, Heidelberg, Infantis, Newport, Typhimurium, Javiana, I 4,[5],12:i:-, Muenchen, Saintpaul, Montevideo, Braenderup, Oranienburg, and Thompson.

5. The method of claim 1, wherein the primer pair comprises nucleotide sequences selected from the group consisting of SEQ ID NOS: 1 and 2, SEQ ID NOS: 3 and 4, SEQ ID NOS: 5 and 6, SEQ ID NOS: 7 and 8, SEQ ID NOS: 9 and 10, SEQ ID NOS: 11 and 12, SEQ ID NOS: 13 and 14, SEQ ID NOS: 15 and 16, SEQ ID NOS: 17 and 18, SEQ ID NOS: 19 and 20, SEQ ID NOS: 21 and 22, SEQ ID NOS: 23 and 24, SEQ ID NOS: 25 and 26, and SEQ ID NOS: 27 and 28.

6. The method of claim 1, wherein the nucleic acid probes comprises nucleotide sequences selected from the group consisting of SEQ ID NOS: 31-60.

7. The method of claim 1, wherein the sample is obtained from a subject, a farm animal, a plant, a food product, a processing surface, or water or a swab thereof.

8. A method for serotyping a Salmonella in a sample matrix, comprising:
obtaining a sample from the sample matrix;
isolating total DNA therefrom;
performing an amplification reaction on the total DNA using a plurality of fluorescently-labeled primer pairs selective for all Salmonella serovars to generate fluorescently-labeled serovar DNA amplicons;
hybridizing the fluorescently-labeled serovar DNA amplicons to a plurality of nucleic acid probes each having a sequence complementary to a sequence determinant in the Salmonella DNA that discriminates among the Salmonella serovars, each of said nucleic acid probes attached at a specific position on a microarray support;
washing the microarray support at least once;
imaging the microarray support to detect at least one fluorescent signal from the hybridized fluorescently-labeled serovar DNA amplicons; and
correlating the specific position of the fluorescent signal on the microarray support to a specific Salmonella serotype.

9. The method of claim 8, wherein the sample matrix is processed with an enrichment culture or without an enrichment culture.

10. The method of claim 9, wherein the sample matrix is processed without the enrichment culture, said sample comprising a rinsate of the sample matrix or a swab of the sample matrix.

11. The method of claim 8, wherein the Salmonella serotype is Salmonella enterica Enteritidis, Salmonella enterica Heidelberg, Salmonella enterica Infantis, Salmonella enterica Newport, Salmonella enterica Typhimurium, Salmonella enterica Javiana, Salmonella enterica I 4,[5],12:i:-, Salmonella enterica Muenchen, Salmonella enterica Saintpaul, Salmonella enterica Montevideo, Salmonella enterica Braenderup, Salmonella enterica Oranienburg, or Salmonella enterica Thompson.

12. The method of claim 8, wherein the plurality of primer pairs comprises nucleotide sequences of SEQ ID NOS: 1 and 2, SEQ ID NOS: 3 and 4, SEQ ID NOS: 5 and 6, SEQ ID NOS: 7 and 8, SEQ ID NOS: 9 and 10, or SEQ ID NOS: 11 and 12, SEQ ID NOS: 13 and 14, SEQ ID NOS: 15 and 16, SEQ ID NOS: 17 and 18, SEQ ID NOS: 19 and 20, SEQ ID NOS: 21 and 22, SEQ ID NOS: 23 and 24, SEQ ID NOS: 25 and 26, and SEQ ID NOS: 27 and 28.

13. The method of claim 8, wherein the plurality of nucleic acid probes comprise nucleotide sequences of SEQ ID NOS: 31-60.

14. The method of claim 8, wherein the sample is obtained from a subject, a farm animal, a plant, a food product, a processing surface, or water or a swab thereof.

15. A method for testing a food product for the presence of Salmonella, comprising:
obtaining a selective media enrichment of a food matrix associated with the food product;
extracting a bacterial pool therefrom;
isolating total DNA from the bacterial pool;
performing at least one amplification reaction on the total DNA using at least one fluorescently-labeled primer pair selective for at least one Salmonella sp. serovar gene target and a generic Salmonella sp. marker to generate fluorescently-labeled serovar DNA amplicons;
hybridizing the fluorescently-labeled serovar DNA amplicons to nucleic acid probes each having a sequence complementary to a gene sequence determinant in at least one Salmonella sp. DNA that discriminates among the Salmonella sp. serovars, each of said nucleic acid probes attached at a specific position on a microarray support;
washing the microarray at least once;
imaging the microarray support to detect at least one fluorescent signal from the hybridized fluorescently-labeled serovar DNA amplicons, wherein the specific position of the fluorescent signal and a target gene profile on the microarray support identifies a specific Salmonella sp. serotype in the food product.

16. The method of claim 15, wherein the Salmonella sp. is Salmonella enterica.

17. The method of claim 15, wherein the Salmonella sp. serovar is Salmonella enterica Enteritidis, Salmonella enterica Heidelberg, Salmonella enterica Infantis, Salmonella enterica Newport, Salmonella enterica Typhimurium, Salmonella enterica Javiana, Salmonella enterica I 4,[5],12:i:-, Salmonella enterica Muenchen, Salmonella enterica Saintpaul, Salmonella enterica Montevideo, Salmonella enterica Braenderup, Salmonella enterica Oranienburg, or Salmonella enterica Thompson.

18. The method of claim 15, wherein the plurality of primer pairs comprises nucleotide sequences of SEQ ID NOS: 1 and 2, SEQ ID NOS: 3 and 4, SEQ ID NOS: 5 and 6, SEQ ID NOS: 7 and 8, SEQ ID NOS: 9 and 10, SEQ ID NOS: 11 and 12, SEQ ID NOS: 13 and 14, SEQ ID NOS: 15 and 16, SEQ ID NOS: 17 and 18, SEQ ID NOS: 19 and 20, SEQ ID NOS: 21 and 22, SEQ ID NOS: 23 and 24, SEQ ID NOS: 25 and 26, and SEQ ID NOS: 27 and 28.

19. The method of claim 15, wherein the plurality of nucleic acid probes comprises nucleotide sequences of SEQ ID NOS: 31-60.

20. The method of claim 15, wherein the generic Salmonella sp. marker is invA.

21. The method of claim 15, wherein the food product is a product from a farm animal, a cultivated plant or water used in the raising or cultivation thereof or from a processing surface for the food product.

22. The method of claim 15, wherein the food product is a processed food product.
